(19) United States
(12) Patent Application Publication
(10) Pub. No.: US 2008/0194976 A1
(43) Pub. Date: Aug. 14, 2008

(54) Diaphragm assembly for blood pressure measuring apparatus

(75) Inventors: Raymond A. Lia, Auburn, NY (US); John W. Sims, Weedsport, NY (US); Uwe Valentin, Hechingen (DE); Hansjorg Frei, Diepoldsau (CH)

Correspondence Address: Hiscock & Barclay, LLP, One Park Place, 300 South State Street, Syracuse, NY, 13202-2078, US

(73) Assignee: WELCH ALLYN, INC., Skaneateles Falls, NY (US)

(21) Appl. No.: 11/705,221

(22) Filed: Feb. 12, 2007

(51) Int. Cl. A61B 5/02

(52) U.S. Cl. 600/490; 600/500

(57) ABSTRACT

A blood pressure measuring apparatus includes a gage and sleeve attached to the gage that includes an interior pressure chamber. A diaphragm is disposed within the gage, the diaphragm being responsive to pressure changes in said sleeve, wherein the diaphragm is fabricated from a nickel-beryllium alloy.

### FIELD OF THE INVENTION

The invention relates to the field of pressure measuring instruments, and more particularly to an improved diaphragm/bellows assembly for a blood pressure measuring instrument.

### BACKGROUND OF THE INVENTION

Various forms of pressure measuring apparatus are presently known. Among these apparatus are devices such as sphygmomanometers, which are used to measure the systolic and diastolic blood pressure of a patient. Sphygmomanometers typically include a pneumatic bulb or pump or other means used to inflate a pressure chamber of an attached sleeve that is fitted over a limb (i.e., an arm or a leg) of the patient. A diaphragm or bellows assembly, responsive to changes in fluid pressure of the pneumatic bulb and the sleeve pressure chamber, is positioned within a gage housing that is fluidly connected to the pressure chamber of the sleeve, typically through flexible tubes or hoses. The gage housing includes a movement mechanism acted upon by the diaphragm assembly to produce corresponding movement of a pointer or indicator element relative to a dial face having indicia.

Until recently, movement mechanisms for the above referred to apparatus were typically quite intricate and complex, and were akin in terms of their manufacture and precision to that of Swiss watches. For example, and in one such movement mechanism, a pair of diaphragm springs are attached adjacent opposing ends of a spindle. A bottom end of the spindle is placed in contact with the bellows assembly and a twisted bronze band, perpendicularly disposed at the top end of the spindle, is connected in parallel by a horizontally disposed spring bent part. As the spindle deflects axially in response to the inflation of the bellows, the bent spring part is also caused to deflect, thereby causing the band to twist. The pointer, attached to the bronze band, therefore, is caused to rotate in relation to an adjacent dial face.

Devices, such as the foregoing, included numerous moving and relatively complex components, some or each of which have numerous bearing surfaces. Therefore, such devices had to be manufactured with relatively strict tolerance margins and significant associated costs in terms of both precision and accuracy in order to minimize errors, as well as part failure rates.

In addition, any adjustments required after assembly of the above noted movement mechanisms, such as to null the pointer element or adjust the sensitivity of the device, required substantial tear down or at least some undesired disassembly of the apparatus.

Furthermore, discrete and separate elements are typically required within the instrument housing for independently supporting the movement mechanism and the diaphragm/bellows assembly, respectively, and for defining an expansion chamber for the bellows assembly therebetween.

A more recent and simplified movement mechanism for use in pressure measuring apparatus has been developed by Applicant, as described in U.S. Pat. No. 5,996,829, incorporated by reference in its entirety. This simplified movement mechanism is defined by a vertically disposed axial cartridge having a spirally wrapped ribbon spring in which one end of the spring is mounted to an axially movable elongate shaft and the remaining end of the spring is attached to a fixed portion within the housing, such as a tubular sleeve. A bottom portion of the axially movable elongate shaft is positioned relative to an expandable diaphragm or bellows assembly, wherein subsequent axial translation of the shaft, caused by movement of the diaphragm in response to pressure changes in the connected sleeve, elongates the spirally wound ribbon spring and produces repeatable circumferential movement of the pointer or indicator element that is supported at the top end of the shaft. The above movement mechanism is much more compact and lightweight than those previously known due to its simplified and axial construction. To that end, it was determined that the design of the gage housing, due to the improved movement mechanism, could also be greatly simplified. The corresponding simplified gage housing design permitted direct fluidic connection to the interior chamber of a sleeve without the need for hoses or other intermediate coupling means. This connection is described in U.S. Pat. No. 6,615,666, the entire contents of which are herein incorporated by reference. Other connections were also made possible due to the convenience, light weight and portability afforded by the simplified gage housing design.

Though Applicants have been able to create enhancements and improvements to blood pressure apparatus, it should be noted that medical and other standards have constricted the ability to use both analog and digital pressure measuring devices, particularly in Europe, such as BSEN 1060-1:1996 as amended 8 Oct. 2002, but also including other standards, such as ANSI/AAMI SP10:2002, among others in which repeatability and drift are factors that contribute to errors, including those errors that are based upon temperature. That is to say, these standards prevent the use of pressure measuring devices that produce variable results based on typical changes in temperature that are encountered by a patient (between approximately 50° F.-104° F.). These standards are perhaps more difficult given the direct connective nature of the gage housing within an inflatable sleeve that is attached to the skin of a patient. A key contributor to this error budget is the diaphragm assembly. Heretofore, diaphragms have been constructed of beryllium-copper, which is ideal for manufacture. However, beryllium-copper has a tendency to drift with temperature and is therefore not ideal under the new standards.

It is therefore a need in the field to provide a blood pressure measuring apparatus that permits more accurate and repeatable blood pressure measurements to be made and in which these measurements can be made over a range of temperatures that typify those encountered by a patient and caregiver.

### SUMMARY OF THE INVENTION

Therefore and according to one aspect, there is provided a blood pressure measuring apparatus comprising an inflatable sleeve having an interior chamber, and a gage housing attached to the inflatable sleeve, said gage housing including a movement mechanism having a pointer element and a diaphragm assembly disposed in relation to said movement mechanism that includes at least one movable surface responsive to pressure changes in the interior chamber of said inflatable sleeve, said at least one movable surface of said diaphragm assembly being made from a nickel-beryllium alloy.

According to another aspect, there is provided a gage for a blood pressure measuring apparatus, said gage including a housing that retains a movement mechanism and a diaphragm subassembly having at least one movable surface said at least one movable surface being responsive to pressure changes in an inflatable sleeve and in which said at least one movable surface of said diaphragm is made from nickel-beryllium alloy.

According to yet another aspect, there is provided a diaphragm assembly for a pressure measuring device, said diaphragm assembly including said at least one movable surface that is made from a nickel-beryllium alloy.

One advantage provided is a more thermally dynamically stable pressure measuring apparatus than those of the prior art wherein the diaphragm assembly provides accurate and repeatable movement under pressure loads.

It will be readily apparent that other aspects and advantages are possible as read from the following Detailed Description in conjunction with the accompanying drawings.

### BRIEF DESCRIPTION OF THE DRAWINGS

FIG. 1 is a side elevational view, shown in section, of a pressure measuring device in accordance with a first embodiment;

FIG. 2 is an enlarged sectional view of the pressure measuring device of FIG. 1;

FIG. 3 is a side elevational view, shown partly in section, of a pressure measuring device having a housing according to a second embodiment;

FIG. 4 is a partial sectional view of a pressure measuring device made in accordance with a third preferred embodiment as used with an inflatable blood pressure sleeve;

FIG. 5 is a side elevational view, in section, of a gage housing having a diaphragm assembly made in accordance with a fourth embodiment; and

FIG. 6 is a partial side elevational view, shown partially in section, of a blood pressure measuring apparatus having a diaphragm assembly made in accordance with a fifth embodiment.

### DETAILED DESCRIPTION

The present invention is herein described with reference to several preferred embodiments, each of which specifically relates to blood pressure measuring apparatus. However, it should be evident to one of sufficient skill in the field that certain other variations and modifications could be made utilizing the inventive concepts described herein, as well as alternative applications other than blood pressure measurement, including use, for example, in barometers, pressure vessel indicators, pressure sensitive switches, valves, and literally any industrial or medical device requiring a pressure responsive element. Furthermore and throughout the course of the following discussion, terms such as “upwardly,” “downwardly,” “upper,” “lower,” “top,” “bottom,” “vertically,” “horizontally,” and the like are used to provide a frame of reference with regard to the accompanying drawings. These terms, however, should not be treated as limiting except where specifically indicated herein.

A number of terms are also used herein that require definitions. To that end, “gearless” as used herein refers to any movement mechanism disposed within a gage or gage housing that does not include a gear or gear-like element. “Hoseless” as used herein refers to a direct connection between a gage housing and an inflatable sleeve of a pressure measuring apparatus without any intermediate hoses or other coupling means therebetween. “Connected” as used herein refers to state of being reversibly (e.g. releasably) or irreversibly joined in direct contact.

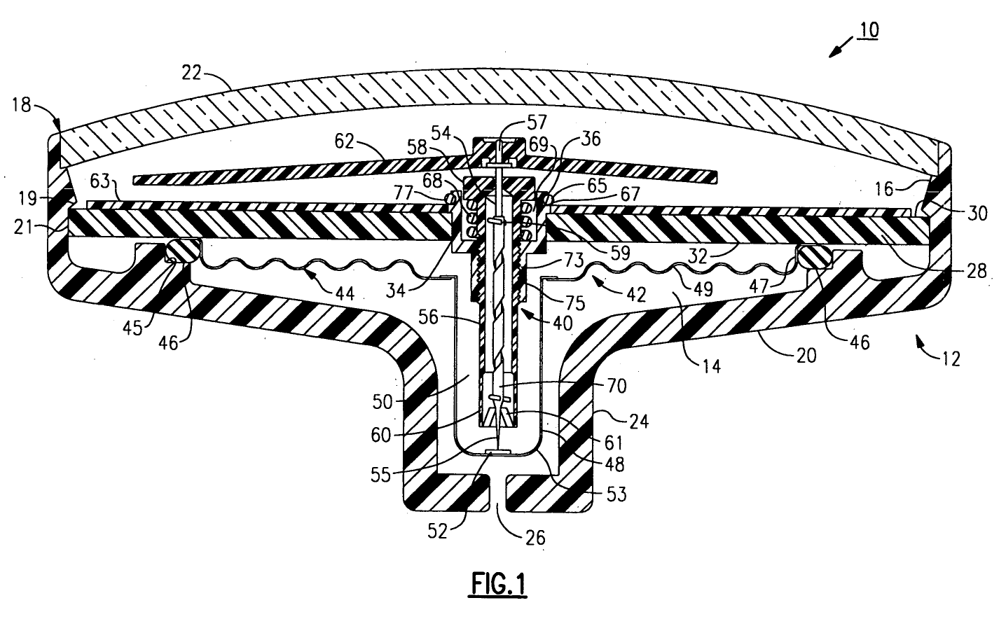

Referring to FIG. 1, there is shown a blood pressure measuring device or apparatus 10 made in accordance with a first embodiment. The device 10 includes a substantially cylindrical gage housing 12 having an interior cavity 14 defined by a circumferential inner wall 16, an open top end 18, and a bottom end 20. A viewing window or bubble 22, made from glass, plastic, or other suitable transparent material, is attached in a known manner to the open top end 18 of the gage housing 12. The bottom end 20 of the gage housing 12 has a diameter that inwardly tapers from a major portion to a narrow downwardly extending portion 24, including a bottom opening 26 that serves as an inlet port for admitting a fluid. According to this embodiment, the diameter of the narrow extending portion 24 is about one third of the diameter of the major portion of the housing 12, though this parameter can be suitably varied, depending upon the application.

The interior cavity 14 of the gage housing 12 is sized for retaining a number of component parts, including a horizontally disposed support plate 28. The support plate 28 is a generally planar member made from a structural material, such as brass or stainless steel or other rigid materials, and having opposing top and bottom facing sides 30, 32, as well as a central through opening 34. A press-fitted or otherwise suitably attached or integral sleeve 36 attached to the top facing side 30 of the support plate 28 extends into the central through opening 34 of the support plate and is used for retaining a movement mechanism 40, the latter being described in greater detail below.

The circumferential inner wall 16 of the gage housing 12 further includes a reflexed portion 19 that is sized for supporting an outer edge 21 of the horizontal support plate 28 immediately therebeneath and at a predetermined height within the gage housing 12. The central through opening 34 is shown as being substantially aligned with the bottom opening 26 of the housing 12, but this particular alignment is not critical to the workings of the device and therefore can be suitably varied.

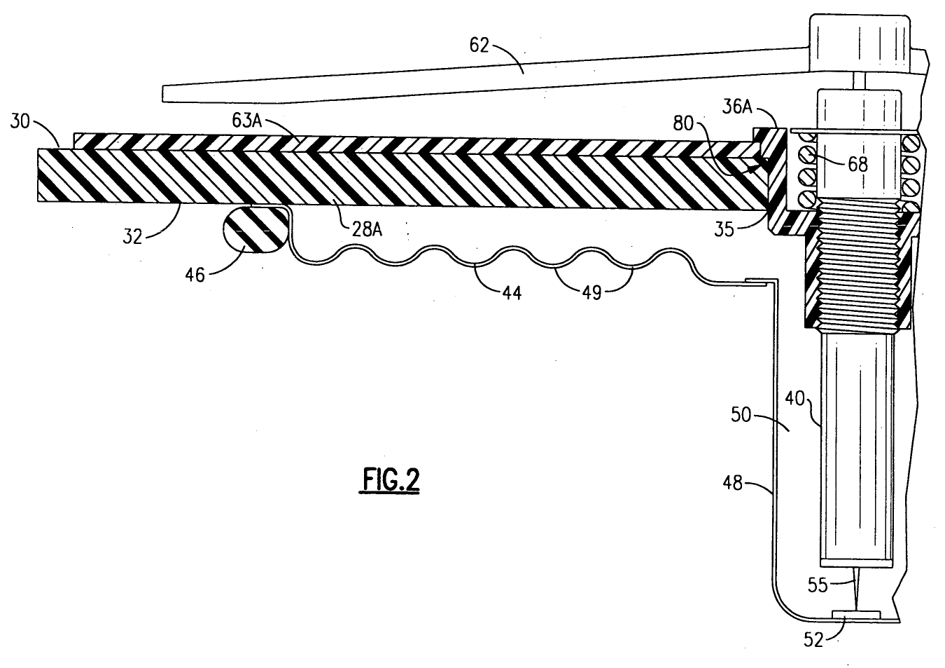

Referring to FIGS. 1 and 2, a diaphragm subassembly 42 includes a flexible diaphragm 44, which is non-fixedly attached to the bottom facing side 32 of the horizontal support plate 28. The diaphragm 44 is fabricated from an alloy that is substantially comprising a combination of nickel-beryllium that has been heat treated by a precipitation hardening process for extended fatigue life, wherein the diaphragm is substantially horizontally planar with the exception of a plurality of wave-like surfaces 49. According to this embodiment, the composition of the diaphragm 44 is approximately 97 weight percent nickel, approximately 2 weight percent beryllium and the remaining approximately 1 weight percent being a combination of titanium whereas the prior art diaphragms are manufactured in weight composition with approximately 97⁺ percent copper, about 2 percent beryllium and the remaining approximately 1 percent having other (i.e., one or a combination of trace materials including aluminum, iron, silicon, titanium, manganese, molybdenum, tungsten and chromium) also formed using a precipitation hardening process. The improved diaphragms produce stiffness changes of nearly zero between 0° C. and 40 ° C. while the prior art diaphragms produce about a one percent stiffness change over the same temperature range. The wave-like surfaces 49 in conjunction with the material properties and thickness are formed to match the diaphragm stroke characteristics to the mechanical movement mechanism 40 of the gage to achieve the desired linearity. According to this design, an outer edge 47 of the diaphragm 44 is clamped by an O-ring 46 disposed on a circumferential ledge 45 extending upwardly from the bottom end 20 of the gage housing 12. The O-ring 46 not only supports the diaphragm 44 in place, but also provides a fluid-tight seal for the bottom of the interior cavity 14 of the gage housing 12.

The centermost portion of the substantially horizontally planar diaphragm 44 further includes a downwardly extending section, hereinafter referred to as the pan 48, which is soldered or otherwise fixed or even integral with the remainder of the diaphragm 44. Like the remainder of the diaphragm 44, the pan 48 is also fabricated from a heat treated nickel beryllium alloy. The pan 48 is a hollow cylindrical section extending into the downwardly extending portion 24 of the gage housing 12, when assembled, and includes a cavity 50 having a width dimension that is substantially equal to that of the press-fitted sleeve 36. A lower end 53 of the pan 48 includes a interior contact surface 52 that is hardened. The contact surface 52 is made from sapphire or hardened steel or similar material that is attached by adhesive or other means to the pan 48.

Referring specifically to FIG. 1, the movement mechanism 40 according to this embodiment includes an axially displaceable shaft member 54 that is wholly enclosed within a hollow tubular member 56, with the exception of protruding top and bottom ends 57, 55, respectively. A thin flexible ribbon-like spring member 70 is fixedly attached at one end 61 adjacent a bottom end of the tubular member 56 to a fixed portion of the tubular member and at an opposite remaining end 59 to the axially displaceable shaft member 54 around which the ribbon spring member 70 is helically or spirally wound. The hollow tubular member 56 includes a set of external threads 73 extending over an upper portion of the length thereof that engage corresponding internal threads 75 provided in the press-fitted sleeve 36. The ribbon spring member 70 is preferably fabricated from beryllium copper, though spring steel, or other similar material could also be utilized to produce the desired effect.

The hollow tubular member 56 according to this embodiment includes an integral top cap portion 58 having a diameter which is larger than that of the remainder of the member, the cap portion having a shoulder which bears against a biasing spring 68 disposed within an annular recess 69 of the press-fitted sleeve 36. The top cap portion 58 and the biasing spring 68 are used according to this embodiment to adjust the overall sensitivity of the movement mechanism 40, although other systems can also be utilized for adjustment purposes.

When correctly positioned, the majority of the movement mechanism 40 extends beneath the horizontal support plate 28 and into the cavity 50 defined within the pan 48, which is already positioned in the downwardly extending portion 24 of the gage housing 12. In this position, the extending bottom end 55 of the shaft member 54 is proximate to the hardened contact surface 52.

Still referring to FIG. 1, a dial face 63 having measuring indicia (not shown) is attached to the top facing side 30 of the horizontal support plate 28 through a center opening which is sized to fit over the press-fitted sleeve 36. An O-ring 65 disposed in a slot 67 of the tubular sleeve 36 engages an inner edge of the dial face 63 with an indicating member 62 being mounted to the protruding top end 57 of the shaft member 54.

In operation, changes in the pressure of incoming fluid (in this example, air, from the interior chamber of an inflatable sleeve (not shown) wrapped about the limb of a patient) entering the bottom opening 26 of the housing 12 cause corresponding movements of the movable surfaces of the diaphragm 44. That is, the seal provided onto the outer edge 47 of the diaphragm 44 by the O-ring 46 clamping against the top face of the housing ridge 45 prevents air from further penetrating into the interior cavity 14. Therefore, the increase in pressure causes axial movement of the pan 48 of the diaphragm 44 characterized by the wave-like surfaces 49 with the hardened interior contact surface 52 being caused to push upwardly against the bottom end 55 of the axially displaceable shaft member 54. As a result of the corresponding upward movement of the diaphragm 44, the top end of the ribbon spring member 70 is caused to extend relative to the fixed bottom end 61 of the spring member, which is fixedly attached to the bottom end of the tubular member 56. This extension causes the shaft member 54 to rotate about its linear axis. The rotation of the axially displaceable shaft member 54, therefore, causes a corresponding circumferential movement of the indicating member 62 attached to the top end 57 of the shaft member 54 relative to the measuring indicia (not shown) on the dial face 63.

Due to the nickel-beryllium construction of the diaphragm 44, the associated movements of the diaphragm 44 are thermally stable dynamically, even with anticipated changes in temperature (10° C.-40° C.), as opposed to diaphragms typically constructed from copper or beryllium-copper. FIG. 2 illustrates a similar, but enlarged view of the gage housing 12 and diaphragm assembly 42.

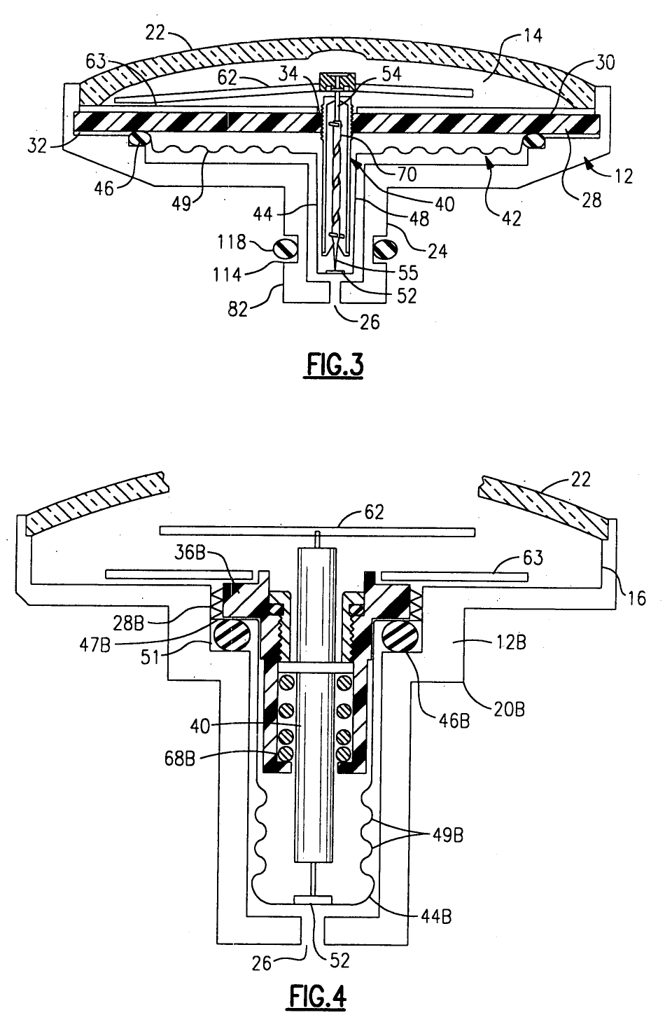

A design made in accordance with a second embodiment is illustrated in FIG. 3. Similar parts are herein labeled with the same reference numerals for the sake of clarity. As in the preceding, the apparatus includes a gage housing 12 having an interior cavity 14 that is appropriately sized for retaining a diaphragm assembly 42. Also and like the preceding, the diaphragm assembly 42 is defined by a diaphragm 44 that is fabricated from a nickel beryllium alloy that is preferably heat treated and is further defined by a plurality of movable wave-like surfaces 49 that are disposed along a substantially horizontal planar portion, as well as a downwardly extending portion or pan 48 that is soldered or otherwise attached to the remainder of the diaphragm 44 or alternatively is integral therewith. As in the preceding, the diaphragm 44 according to this embodiment is made from a nickel-beryllium alloy having a composition of about 97⁺ percent nickel, about 2 percent beryllium and the remaining approximately 1 percent being one or a combination of trace materials including aluminum, iron, silicon, cobalt, titanium, tungsten, molybdenum, chromium and manganese.

The apparatus according to this embodiment further includes a substantially horizontally disposed planar support plate 28, the gage housing 12 further having a downwardly extending narrowed portion 24 corresponding with that of the pan 48. A movement mechanism 40 is disposed through a central opening 34 defined in the horizontal support plate 28 such that the bottom end 55 of an axially displaceable shaft 54 of the mechanism is disposed in proximity to a hardened interior contact surface 52 located at the bottom of the interior of the pan 48 of the diaphragm assembly 42. The diaphragm 44 in the meantime is attached, but is not sealed, to the bottom facing side 32 of the horizontal support plate 28. The interior cavity of the gage housing 12 below the support plate 28 is sealed using an O-ring 46 disposed on a circumferential ledge of the housing in which the ends of the diaphragm 44 are retained between the O-ring and the bottom side 32 of the support plate 28.

Pressure changes in an inflatable sleeve (not shown) cause a fluid, such as air from an interior chamber of the sleeve (also not shown in this view), to enter the gage housing 12 through a bottom opening 26. The preceding causes corresponding movement of the movable surfaces of the diaphragm 44, causing deflection of the hardened interior surface 52 of the pan 48 against the lower end 55 of the axially displaceable shaft 54, to thereby cause rotation of the shaft 54 by means of an attached ribbon spring member 70, in the manner previously described. Like the preceding, the diaphragm 44 in accordance with this apparatus design is more thermally stable dynamically over changes in temperature based on its nickel-beryllium construction, and therefore produces more repeatable movement.

According to this particular embodiment, the apparatus also includes a docking hub 82 that is provided on the exterior of a narrow downwardly extending portion 24 of the gage housing 12. The hub 82 includes a circumferential groove 114 that is sized for retaining an O-ring 118 or other similar sealing element. For example, the docking hub 82 can utilize pipe or other form of known threads (not shown). The docking hub 82 provides adequate modification to allow the apparatus to be attached to other existing pressure device housings having pressure sources, for example, those manufactured by Welch Allyn, Inc. of Skaneateles Falls, N.Y., among others. In passing, it should be noted that the position of the bottom opening 26 of the housing 12 is not essential; that is, incoming fluid can enter the gage housing 12 from either a horizontally or otherwise disposed port, so long as the opening is beneath the seal that is provided by the O-ring 118.

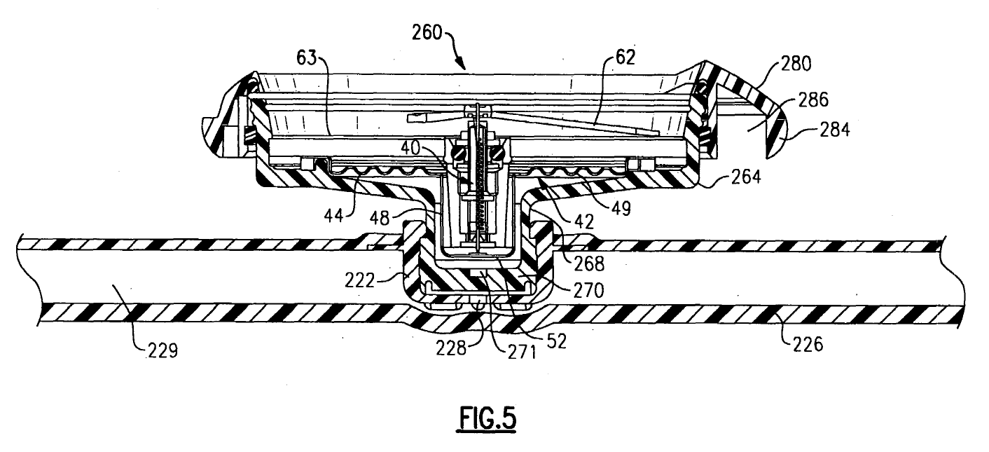

Variations of the above design are possible. For example and referring to FIG. 4, a third embodiment of a gage housing 12B includes a contained diaphragm 44B, which is also made from a heat treated nickel beryllium alloy, but is constructed with a substantially vertical profile having an overall width dimension that is considerably narrower than those that have been previously described. As a result of the design of this diaphragm 44B, a horizontal support plate is not required.

As in the preceding embodiments, an outer edge 47B of the diaphragm 44B is sealed using an O-ring 46B or other sealing member that effectively clamps the outer edge to a shoulder of a press-fitted sleeve 36B. A movement mechanism 40, constructed in the manner previously described, is disposed essentially through a center opening in the press-fitted sleeve 36B and threaded into engagement therewith. The majority of the movement mechanism 40 is disposed within the interior cavity defined by the essentially vertical diaphragm 44B. This movement mechanism 40 can be that as previously described or other variation. This particular diaphragm 44B, like that of the preceding embodiments, is fabricated from a heat treated nickel-beryllium alloy having a composition as described previously and includes a plurality of vertically disposed wave-like movable surfaces 49B that are responsive to pressure variations from an incoming fluid entering the gage housing 12B through a lower or bottom opening 26. This movement is imported to the movement mechanism 40 via a hardened surface 52 engaging with an causing corresponding axial movement relative to the movement mechanism to cause circumferential movement of a pointer element 62 attached to the movement mechanism relative to indicia (not shown) provided on a dial face 63. Adjustments to control the sensitivity of the movement mechanism 40, for example, using a biasing spring 68B, ate performed in the manner previously described.

Overall, the design of the instant embodiment defines a very shallow profile for the upper portion of the gage housing 12B. Though not shown in this view, the bottom end 20B of the gage housing 12B can be used as a docking hub to secure the gage housing into other gage housings (not shown), either as a retrofitted or as a new assembly as previously described. As further described herein, this docking hub can also permit direct hose-free connection between a gage housing and an inflatable blood pressure sleeve (not shown).

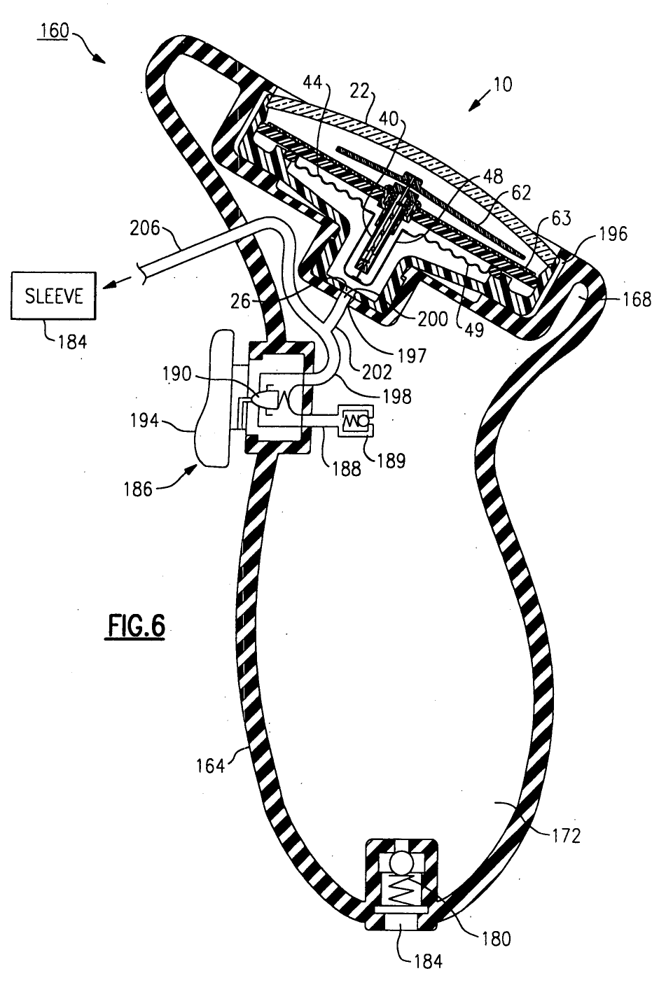

Referring to FIG. 5, there is shown a gage housing 260 defined according to a fourth embodiment. As in the preceding, similar parts are labeled with the same reference numerals for the sake of clarity. In this embodiment, the gage housing 260 includes an upper housing portion 264 and a narrowed lower housing portion 268 having an engagement end 270 that directly mates with a socket 222 that is formed in a blood pressure sleeve 226 (partially shown). One typical sleeve design is described more completely in U.S. Pat. No. 6,036,718, the entire contents of which are herein incorporated by reference though other variations are possible, this sleeve being exemplary. The upper housing portion 264 according to this embodiment is defined by a substantially elliptical cylindrical cross section, though the upper housing portion can assume other shapes, such as circular or polyhedral. To that end, it should be noted that literally any shape or geometries could be contemplated.

The engagement end 270 engages the socket 222 of the interior of the sleeve 226 in a snap fitting to provide both a mechanical as well as a fluidic interconnection with the sleeve 226. The engagement end 270 includes a bottom end opening 271 that permits direct (hoseless) fluid communication with the sleeve 226, through a socket opening 228, extending to the sleeve interior 229.

The gage housing 260 retains a movement mechanism 40 that includes an axially movable shaft member 54, FIG. 1, as previously described and further includes a helically wound spring member 70, FIG. 1, one end of which is attached to the shaft member and the remaining end being attached to a fixed portion of the housing. Further details relating to an exemplary movement mechanism are provided in U.S. Pat. No. 5,966,829, previously incorporated herein.

A diaphragm assembly 42 is disposed in relation to the movement mechanism 40, the diaphragm assembly including a diaphragm 44 made from a heat treated nickel-beryllium alloy having a composition as described previously. The diaphragm assembly 42, like the preceding includes a diaphragm 44 having a horizontal planar portion that includes a plurality of wave-like surfaces 49 as well as an extending pan 48 sized to extend into the narrowed lower portion 268 of the gage housing 260 including an interior cavity that retains a substantial portion of the movement mechanism 40.

According to the instant embodiment, a rubberized guard member 280 is press fitted over the exterior periphery of the upper housing portion 264, the guard member according to this embodiment including a radially extending portion 284 which when attached extends from the outer edge of the upper housing portion 264 and similarly provides a cushioning air gap 286 which creates a discontinuity, in fact a buffer, which insulates the housing 260 from impact loads when the housing is dropped. Similar air gaps 288 are provided above the viewing window, as defined in an axially extending portion 290, in order to provide additional protection against shock or impact loads. Details relating to those latter features are described in U.S. Pat. No. 6,615,666, incorporated by reference herein.

As in the preceding, the diaphragm 44 is attached between a support plate and a seal member. In this instance, the upper housing portion 264 includes a radial protrusion 265, as opposed to use of an O-ring, that retains an outer edge of the diaphragm 44 between the protrusion and the support plate and prevents fluid from entering the upper portion 264 of the housing 260. In operation, fluid (i.e., air) escapes the sleeve interior 229 through socket opening 228 and enters the engagement end 270 of the housing 260 through opening 271 into the lower portion 268. The movable surfaces of the diaphragm 44 are caused to move based on the changes in pressure due to the entering fluid and as in the preceding, the hardened interior surface 52, FIG. 1, of the pan 48 engages the lower end of the axially movable shaft 54, FIG. 1. The shaft 54, FIG. 1, is caused to translate against the bias of the helically wound spring 70, FIG. 1, and this causes the shaft to rotate causing circumferential movement of the indicating member in relation to the dial face.

The direct connection between the socket which is preferably elastomeric and the engagement end 270 of the housing 260 produces a direct hoseless connection between an inflatable sleeve 226 and the gage housing. The sleeve 226 is wrapped about the patient, but due to the dynamic thermal stability provided by the diaphragm 44, there is no appreciable drift caused by temperature variations, such as between ambient and body temperature differences.

It can be seen that the herein described diaphragm assembly can be included in other device variations, such as shown in FIG. 6. As in the preceding, similar parts are herein labeled with the same reference numerals, for the sake of clarity. An integrated blood pressure measuring apparatus 160 includes an elastomeric or enclosure sleeve 164 that includes an upper portion 168 and a contiguous lower portion 172. A pneumatic bulb (not shown) can be provided within the lower portion 172 of the elastomeric sleeve 164 or alternatively, the sleeve itself can provide the means for pneumatically inflating a blood pressure cuff (shown diagrammatically herein as 184). A one way valve 182 provided at the bottom of the lower portion 172 of the sleeve 164, according to this embodiment, permits air to enter the interior of the sleeve 164 when squeezed.

A trigger assembly 186 disposed along a wall of the sleeve 164 includes a bleed valve 190 that can be opened by means of an actuable trigger 194 provided on the exterior of the sleeve 164. The trigger 194 is biasedly connected to the bleed valve 190 through conventional means such as a spring (not shown). A hose 188 extends into the trigger assembly 186, the hose including a valve 189 that permits air from the interior of the sleeve 164 to pass into the hose 188. Another hose 198 extends from the trigger assembly 186, splitting into sections 202, 206 respectively. Section 206 extends from the device 160 to the inflatable cuff 184 and is connected to a port (not shown) which is in fluid communication with the interior of the sleeve.

The sleeve 160 further includes a retaining pocket 196 that is preferably smaller in diameter than that of a gage housing 12 like that previously described in FIG. 1 to provide secure engagement with the exterior thereof. The gage housing 12 includes a movement mechanism 40, such as described by previously incorporated U.S. Pat. No. 5,966,829, that permits circumferential movement of an indicating member 62 relative to a dial face 63. In addition, the gage housing 12 is defined by an upper housing portion and a narrowed lower housing portion that is fitted into a narrowed bottom portion of the retaining pocket 196. The bottom portion of the retaining pocket 196 includes the port 200 that enables fluid connection between the hose 197 that provides a fluid path through the port and an aligned opening 197 into the interior of the gage housing 12 through an opening 26 provided in the lower portion 24 of the gage housing 12.

In operation, the lower portion 172 of the sleeve 164 is squeezed permitting air to enter the interior of the sleeve and into hose 188 through check valve 187. Initially, the bleed valve 190 is closed, therefore air is directed through the trigger assembly 186 and through hose 198 and hose sections 202, 206 to the interior of the gage housing 12 and the inflatable sleeve or cuff 184, shown schematically, permitting inflation of the latter.

Opening of the bleed valve 190 is accomplished through use of the trigger 194 which permits deflation of the sleeve 184 whereupon a blood pressure measurement can be made.

Pressure changes in the chamber (not shown) of the attached inflatable cuff 184 are communicated to the movement mechanism 40 by corresponding movement of the pan 48 and movable surfaces of the diaphragm 44, thereby causing movement of the axially displaceable shaft member of the movement mechanism to produce resulting circumferential movement of the indicating member 62.

### PARTS LIST FOR FIGS. 1-16

10 blood pressure measuring device or apparatus
12 gage housing
12B gage housing
14 interior cavity
16 circumferential inner wall
18 open top end
19 reflexed portion
20 bottom end
20B bottom end
21 outer edge (support plate)
22 bubble or viewing window
24 downwardly extending portion
26 bottom opening
28 horizontal support plate
28A horizontal support plate
30 top facing side
32 bottom facing side
34 central through opening
36 sleeve
36A sleeve
36B sleeve
40 movement mechanism
42 diaphragm subassembly
44 diaphragm
44B diaphragm
45 circumferential ridge
46 O-ring
46B O-ring
47 outer edge
47B outer edge
48 pan
49 wave-like surfaces
40B wave-like surfaces
50 cavity
51 cavity
52 contact surface
53 lower end
54 axially displaceable shaft member
55 bottom end
56 tubular member
57 top end
58 top cap portion
59 end-ribbon spring
61 end-ribbon spring
62 indicating member
63 dial face
63A dial face
65 O-ring
66 threads
67 slot
68 biasing spring
68B biasing spring
69 recess
70 ribbon spring member
72 one end
73 threads
75 threads
80 shoulder
82 docking hub
114 circumferential groove
118 O-ring
160 blood pressure measuring apparatus
164 sleeve, elastomeric
168 upper portion
172 lower portion
180 one way valve
184 sleeve, inflatable or cuff
186 trigger assembly
188 hose
189 check valve
190 bleed valve
194 trigger
196 pocket
197 opening
198 hose
200 port
202 hose
206 hose
222 socket
224 end opening
226 blood pressure sleeve or cuff
228 socket opening
229 sleeve interior
260 gage housing
264 upper housing portion
268 narrowed lower housing portion
270 engagement end
271 end opening
280 rubberized guard member
284 radially extending portion
286 air gap

Although the present invention has been described herein with reference to details of currently preferred embodiments, it is not intended that such details be regarded as limiting the scope of the invention, except as and to the extent that they are included in the following claims—that is, the foregoing description of the present invention is merely illustrative, and it should be understood that variations and modifications can be effected without departing from the scope or spirit of the invention as set forth in the following claims. Moreover, any document(s) mentioned herein are incorporated by reference in their entirety, as are any other documents that are referenced within the document(s) mentioned herein.

We claim:

1. A blood pressure measuring apparatus comprising:
a gage;
an inflatable sleeve attached to said gage that is wrappable about a limb of a patient, said sleeve including an interior chamber;
a diaphragm assembly disposed within said gage, said diaphragm assembly including a diaphragm including at least one movable surface that is responsive to pressure changes in said interior chamber of said inflatable sleeve, wherein said diaphragm is fabricated from a nickel-beryllium alloy to improve dynamic thermal stability of said apparatus.

2. An apparatus as recited in claim 1, wherein said gage is directly attached to said sleeve.

3. An apparatus as recited in claim 2, wherein said gage is releasably attached to said inflatable sleeve.

4. An apparatus as recited in claim 2, wherein said gage includes an engagement portion having an opening and said sleeve includes a socket disposed on said exterior having an opening extending into said pressure chamber.

5. An apparatus as recited in claim 1, wherein said gage is attachable to an integrated pneumatic apparatus.

6. A gage for a blood pressure measuring apparatus, said gage comprising:
a housing;
a movement mechanism; and
a diaphragm assembly, said diaphragm assembly including at least one movable surface responsive to pressure changes and in which said at least one movable surface is made from a nickel-beryllium alloy to improve dynamic thermal stability of said gage.

7. A diaphragm assembly for a pressure measuring device, said diaphragm assembly including at least one movable surface responsive to pressure changes and made from a nickel-beryllium alloy, said diaphragm assembly being dynamically stable over a range of temperature changes.

8. A diaphragm assembly as recited in claim 7, wherein said assembly is stable dynamically over a range between about 10° C. and 40° C.
